(19) United States
(12) Patent Application Publication
(10) Pub. No.: US 2002/0120169 A1
(43) Pub. Date: Aug. 29, 2002

(54) Process facilitating the regeneration of a catalyst based on a zeolite used in an acylation reaction, catalyst and use

(76) Inventors: Michel Spagnol, Lyon (FR); Laurent Gilbert, Paris (FR); Anne-Marie Le Govic, Paris (FR)

Correspondence Address: RHODIA INC., CN-7500, 259 Prospect Plains Road, Cranbury, NJ, 08512, US

(21) Appl. No.: 10/081,241

(22) Filed: Feb. 21, 2002

Related U.S. Application Data
(62) Division of application No. 08/702,723, filed on Dec. 9, 1996, now abandoned.
Application No. 08/702,723 is a 371 of international application No. PCT/FR96/00054, filed on Jan. 12, 1996.

(30) Foreign Application Priority Data
Jan. 13, 1995 (FR) 95/00346

(51) Int. Cl. C07C 45/46; B01J 29/00

(52) U.S. Cl. 568/316; 568/319; 502/20; 502/74

(57) ABSTRACT

The present invention relates to a process facilitating the regeneration of a catalyst based on a zeolite, employed in an acylation reaction.

Another subject of the invention is a process for acylation of an aromatic ether, comprising, in a preferred alternative form, an additional stage of regeneration of the catalyst.

The process of the invention, which makes it possible to regenerate more easily a catalyst based on a zeolite, employed in an acylation reaction, is characterized in that the zeolite is modified by addition of an effective quantity of at least one metallic element M chosen from the elements of group 8 of the Periodic Classification of the elements.

The present invention relates to a process facilitating the regeneration of a catalyst based on a zeolite, employed in an acylation reaction.

Another subject of the invention is a process for acylation of an aromatic ether, comprising, in a preferred alternative form, an additional stage of regeneration of the catalyst.

More particularly, the invention relates to a process for acylation in the position para to the alkoxy group carried by the aromatic nucleus.

The invention applies especially to the preparation of alkoxyaromatic alkyl ketones.

Conventional processes for acylation of aromatic compounds, especially of phenol ethers, make use, as acylating reactant, of a carboxylic acid or of one of its derivatives such as acid halide, ester or anhydride.

The reaction is generally conducted in the presence of a catalyst of the Lewis acid type (for example AlCl₃) or of the Brönsted acid type (H₂SO₄, HF, and the like).

The use of zeolites as acylation catalysts has been proposed for some ten years.

Thus, the vapour phase reaction of an aromatic compound (veratrole) with a carboxylic acid derivative in the presence of a zeolite in H form, such as mordenite, faujasite and ZSM-5, was described in EP-A-0279322.

U.S. Pat. No. 4,960,943 also describes a process of acylation especially of anisole, in the presence of zeolites which have a pore size of at least 5 angstroms and which correspond to the following formula: M_{m/z}[mME¹O₂.nMe²O₂].qH₂O in which M is an exchangeable cation, z is the valency of the cation and Me¹ and Me² denote the elements of the anionic lattice, n/m is a number between 1-3000, preferably 1-2000 and q denotes the adsorbed water.

The development of zeolites on an industrial scale is limited by the fact that zeolites are deactivated relatively quickly. In fact, the deactivation is doubtless attributed to poisoning of the catalyst by the heavy organic products formed during the reaction, which causes a lowering in the degree of conversion of the starting substrate, thus resulting in a drop of output efficiency.

The problem of reactivation of the catalyst then arises, which often involves a tedious regeneration procedure at high temperature (temperature above or equal to 500° C.) requiring specific equipment.

The aim of the invention is to provide a process allowing the abovementioned disadvantages to be avoided.

The subject of the present invention is precisely a process facilitating the regeneration of a zeolite-based catalyst employed in an acylation reaction, characterized in that the zeolite is modified by addition of an effective quantity of at least one metallic element M chosen from the elements of group 8 of the Periodic Classification of the elements.

It has been found that the fact of doping the zeolite with a transition element of group 8 improves the regeneration of the catalyst, which can be carried out, preferably, by simple oxidation with air at a temperature which is not particularly high.

In a preferred alternative form of the invention the process consists in choosing from the metals of group 8, more particularly those of the platinum group.

“The platinum group” commonly denotes the group of the following six metals: ruthenium, rhodium, palladium, osmium, iridium and platinum.

It was found, unexpectedly, that the fact of modifying the zeolite with an element of the platinum group, symbolized by M₁, resulted not only in an easier regeneration of the catalyst, but also enabled a good reaction yield and an excellent selectivity of the reaction to be obtained in the reaction of acylation of an aromatic ether.

The zeolites thus modified are outstanding catalysts of acylation of an aromatic ether. The invention therefore also lies in a process for acylation of an aromatic ether, preferably followed by a stage of regeneration of the catalyst.

According to a preferred alternative form of the process of the invention the acylation of the aromatic ether is first of all performed by reacting it with an acylating agent in the presence of an effective quantity of a zeolite modified with at least one element M₁ of the platinum group, as defined, and then, after reaction, the regeneration of the catalyst is carried out by heat-treating it with a gas containing molecular oxygen.

This preferential embodiment of the process of the invention not only makes it possible to have good performance in respect of the acylation reaction but it is also possible to regenerate the catalyst very easily.

In accordance with the process of the invention the zeolite employed as catalyst in an acylation reaction is modified with a metallic element chosen from the elements of group 8 of the Periodic Classification of the elements.

For the definition of the elements reference is made below to the Periodic Classification of the elements as published in the Bulletin de la Société Chimique de France, No. 1 (1966).

Those involved are more particularly iron, nickel, ruthenium, rhodium, palladium, osmium, iridium and platinum.

A mixture of the said elements may also be employed.

Palladium or platinum may be mentioned as metallic elements which are preferably used.

The content of metallic element M in the zeolite is generally such that the percentage by weight of the metal relative to the zeolite is between 0.1% and 25%, preferably between 3 and 15%.

The metallic element M is introduced into the zeolite by a cation exchange.

“Zeolite” is intended to mean a crystalline tectosilicate of natural or synthetic origin in which the crystals result from the three-dimensional assembly of tetrahedral SiO₄ and TO₄ units, T denoting a trivalent element such as aluminium, gallium, boron, and iron, preferably aluminium.

Zeolites of aluminosilicate type are the most common ones.

Within the crystal lattice zeolites have a system of cavities connected together by channels of a well-defined diameter, which are called pores.

Zeolites may have a one-dimensional, two-dimensional or three-dimensional network of channels.

A natural or synthetic zeolite may be used in the process of the invention.

Examples which may be mentioned of natural zeolites that can be employed are, for example: chabazite, clinoptilolite, erionite, phillipsite and offretite.

Synthetic zeolites are wholly suitable for carrying out the invention.

Examples which may be mentioned of synthetic zeolites with a one-dimensional network are, among others, zeolite ZSM-4, zeolite L, zeolite ZSM-12, zeolite ZSM-22, zeolite ZSM-23 and zeolite ZSM-48.

Examples which may be mentioned of zeolites with a two-dimensional network, which are preferably employed, are zeolite β, mordenite and ferrierite.

Insofar as the zeolites with a three-dimensional network are concerned, zeolite Y, zeolite X, zeolite ZSM-5, zeolite ZSM-11 and offretite may more particularly be mentioned.

Synthetic zeolites are preferably used and, more particularly, the zeolites which are in the following forms:

mazzite with an Si/Al molar ratio of 3.4,

zeolite L with an Si/Al molar ratio of 1.5 to 3.5,

mordenite with an Si/Al molar ratio of 5 to 15,

ferrierite with an Si/Al molar ratio of 3 to 10,

offretite with an Si/Al molar ratio of 4 to 8.5,

zeolites β with an Si/Al molar ratio higher than 8, preferably of between 10 and 35 and, still more preferably, between 12 and 35,

zeolites Y, in particular the zeolites obtained after dealumination treatment (for example hydrotreatment, washing with hydrochloric acid or treatment with SiCl₄), and it is possible to mention more particularly zeolites US-Y with an Si/Al molar ratio higher than 3, preferably between 6 and 60,

zeolite X of faujasite type with an Si/Al molar ratio of 0.7 to 1.5,

zeolites ZSM-5 or aluminium silicalite with an Si/Al molar ratio of 10 to 500,

zeolite ZSM-11 with an Si/Al molar ratio of 5 to 30.

Among all these zeolites the zeolites β and Y are preferably used in the process of the invention.

The zeolites used in the process of the invention are known products described in the literature [cf. Atlas of zeolite structure types by W. M. Meier and D. H. Olson, published by the Structure Commission of the International Zeolite Association (1978)].

Commercially available zeolites may be used or else they may be synthesized according to the processes described in the literature.

Reference may be made to the abovementioned Atlas and, more particularly, for the preparation:

of zeolite L, to the publication by R. M. Barrer et al., Z. Kristallogr., 128, pp. 352 (1969)

of zeolite ZSM-12, to U.S. Pat. No. 3,832,449 and the paper by LaPierre et al., Zeolites 5, pp. 346 (1985),

of zeolite ZSM-22, to the publication by G. T. Kokotailo et al., Zeolites 5, pp. 349 (1985),

of zeolite ZSM-23, to U.S. Pat. No. 4,076,842 and the paper by A. C. Rohrman et al., Zeolites 5, pp. 352 (1985),

of zeolite ZSM-48, to the work by J. L. Schlenker et al., Zeolites 5, pp. 355 (1985),

of zeolite β, to U.S. Pat. No. 3,308,069 and the paper by P. Caullet et al., Zeolites 12, pp. 240 (1992),

of mordenite, to the work by Itabashi et al., Zeolites 6, pp. 30 (1986),

of zeolites X and Y, to U.S. Pat. Nos. 2,882,244 and 3,130,007 respectively,

of zeolite ZSM-5, to U.S. Pat. No. 3,702,886 and the paper by V. P. Shiralkar et al., Zeolites 9, pp. 363 (1989), and

of zeolite ZSM-11, to the work by I. D. Harrison et al., Zeolites 7, pp. 21 (1987).

Zeolites can be employed in various forms in the process of the invention: powder, shaped products such as granules (for example cylinders or beads), tablets and monoliths (honeycomb-shaped blocks), which are obtained by extrusion, moulding, compacting or any other known process type. In practice, on an industrial scale, it is the forms of granules, beads or monoliths that are the most advantageous ones, both from the viewpoint of effectiveness and the viewpoint of convenience in use.

The invention does not rule out the presence of binders employed during the forming of the zeolite, for example aluminas or clays.

Whichever the zeolite chosen, a treatment which makes it acidic is carried out if necessary.

Conventional treatments are used for this purpose.

Thus, the alkali metal cations can be exchanged by subjecting the zeolite to a treatment carried out with aqueous ammonia, thus resulting in an exchange of the alkali metal cation with an ammonium ion, and then the exchanged zeolite may be calcined in order to decompose the ammonium cation thermally and to replace it with an H⁺ ion.

The quantity of aqueous ammonia to be used is at least equal to the quantity needed to exchange all the alkali metal cations for NH₄⁺ ions.

From 10⁻⁵ to 5×10⁻³ mol of aqueous ammonia therefore used per gram of zeolite.

The exchange reaction of the cation which can be exchanged with NH₄⁺ is performed at a temperature which lies between the ambient temperature and the reflux temperature of the reaction medium. The operation takes a few hours and can be repeated.

The zeolite can also be acidified by being subjected to a conventional acid treatment. This treatment may be performed by adding an acid such as especially hydrochloric acid, sulphuric acid, nitric acid, perchloric acid, phosphoric acid and trifluoromethanesulphonic acid.

According to a preferred embodiment the zeolite is acidified by the passage of a volume of acid which has a normality between 0.1 and 2 N per gram of zeolite, of between 10 ml/g and 100 ml/g. This passage can be carried out in a single stage or, preferably, in a number of successive stages.

Conventional techniques, which are known per se, for the preparation of supported metal catalysts can be used to prepare the catalysts involved in the process of the invention. For the preparation of the various catalysts reference may be made, in particular, to the work: J. F. Lepage “Catalyse de contact”, conception, préparation et mise en oeuvre des catalyseurs industrials [“Contact catalysis”, design, preparation and use of industrial catalysts], Technip publishers (1978).

One method of preparation of the catalysts of the invention consists in depositing the element(s) M on the zeolite, precipitating the compounds in a manner known per se and subjecting the contact mass thus obtained to drying and a calcination.

Other methods of preparation are also possible, in particular one method of manufacture of the said catalysts consists in performing the impregnation of the zeolite with an impregnating solution including at least one appropriate compound of the chosen metallic elements, in a volatile solvent, preferably water, and then drying and calcining the contact mass thus obtained.

The zeolite may be chemically modified by impregnation by a dry or wet route.

Thus, one method of preparation consists in performing the dry impregnation of the zeolite with the aid of a solution of at least one compound of a metallic element of group 8.

The impregnation is carried out dry, that is to say that the total volume of solution employed is approximately equal to the total pore volume exhibited by the zeolite. The product obtained is dried and calcined.

More precisely, the dry impregnation consists in adding to a mass m₁ of a powdered product to be impregnated a volume V of an aqueous solution of one or more salts of cations or of anions to be bound onto the surface of the solid. The solution volume V is chosen such that V/m₁ is equal to the water pore volume of the solid to be impregnated.

The concentration C of cations or anions of the impregnating solution is chosen such that the ratio CVM₂/m₁ is equal to the chosen weight percentage of impregnating species bound onto the surface of the product to be impregnated (with M₂=molecular mass of the impregnating species). The solution is added dropwise so as to obtain a homogeneous adsorption.

The product can then be left to stand for a variable period at ambient temperature. The product is then dried according to conventional techniques which are known to a person skilled in the art. The drying is generally conducted at atmospheric pressure or at reduced pressure or by freeze-drying. It may also be calcined.

The wet-route impregnation is done by dispersing the zeolite in an aqueous solution of salts of cations and/or anions to be bound onto the surface of the solid.

This solution may have a concentration of the impregnating species which varies from 10⁻³ M to 10 M.

The pH of the solution may be advantageously adjusted to a value that is at least equal to that of the isoelectric point of the product to be modified in order to bind the cations preferentially (usual case); however, this condition is not essential. Below this isoelectric point it is possible to bind the cations correctly when the associated anions are highly “covalent” in character.

The solution temperature may vary from the ambient to 100° C.

The dispersion is stirred vigorously for a variable time.

The product is then filtered off and optionally washed.

According to both alternative forms as described above a zeolite is obtained on which at least one metallic element M is deposited.

The deposition of several metallic elements on the zeolite can, of course, be carried out successively but, preferably, simultaneously.

In the following stages the modified zeolite is subjected to a drying operation conducted advantageously at a temperature varying from 50° C. to 200° C. for a period ranging preferably from 2 to 12 hours.

Insofar as the calcination operation is concerned, this is conducted at a temperature of between 200° C. and 700° C., preferably between 400° C. and 600° C. for a period varying from 1 to 15 hours, preferably from 2 to 6 hours.

The nature of the compounds supplying the various elements employed for the preparation of the catalysts of the invention is not critical.

The metallic elements M may be supplied in the form of a metal or in the form of an inorganic derivative such as an oxide or a hydroxide. Use may be made of an inorganic salt, preferably nitrate, sulphate, oxysulphate, halide, oxyhalide, silicate or carbonate, or an organic derivative, preferably cyanide, oxalate or acetylacetonate, alcoholate and still more preferably methylate or ethylate, carboxylate and still more preferably acetate. Complexes may also be used, especially chlorinated or cyanated ones of metals M and/or of alkali metals, preferably sodium or potassium or of ammonium.

Examples of compounds capable of being used for the preparation of the catalysts of the invention which may be mentioned are especially:

in the case of iron:

iron(II) bromide

iron(III) bromide

anhydrous or hydrated iron(II) chloride

anhydrous or hydrated iron(III) chloride

iron(II) iodide

iron(III) iodide

iron(II) fluoride

iron(III) fluoride

iron(II) hydroxide

anhydrous or hydrated iron(II) carbonate

iron(II) nitrate

iron(III) nitrate

anhydrous or hydrated iron(II) sulphate

anhydrous or hydrated iron(III) sulphate

iron(III) hydroxysulphate

anhydrous or hydrated iron(II) oxide

anhydrous or hydrated iron(III) oxide

iron(II) orthophosphate

iron(III) orthophosphate

iron(III) oxalate

iron(II) acetate

basic iron(III) acetate

iron(II) citrate

iron(III) citrate

iron(III) acetylacetonate

iron(II) hexamine chloride

in the case of nickel:

nickel(II) bromide

nickel(II) chloride

nickel(II) iodide

nickel(II) carbonate

basic nickel(II) carbonate

nickel(II) cyanide

hydrated nickel(II) nitrate

anhydrous or hydrated nickel(II) sulphate

nickel(II) oxide

nickel(II) hydroxide

nickel(II) acetate

nickel(II) tetramine nitrate

nickel(II) hexamine nitrate

nickel(II) tetramine chloride

nickel(II) hexamine chloride

in the case of ruthenium:

ruthenium(III) chloride

ruthenium(IV) chloride

rutheniumpentafluoride

ruthenium(III) hydroxide

ruthenium(II) oxide

ruthenium(IV) oxide

ruthenium oxychloride treated with aqueous ammonia

Ru₂(OH)₂Cl₄.7NH₃.5H₂O

ruthenium acetate

in the case of palladium:

palladium(II) bromide

palladium(II) chloride

palladium(II) iodide

palladium(II) cyanide

hydrated palladium(II) nitrate

dihydrated palladium(II) sulphate

palladium(II) monoxide, hydrated or otherwise

palladium(IV) dioxide, hydrated or otherwise

palladium(II) acetate

palladium(II) acetylacetonate

ammonium tetrachloropalladate(II)

potassium hexachloropalladate(IV)

palladium(II) tetramine nitrate

palladium(II) dichlorobis(acetonitrile)

palladium(II) dichlorobis(benzonitrile)

palladium(II) dichloro(1,5-cyclooctadiene)

palladium(II) dichlorodiamine

in the case of platinum:

platinum(II) bromide

platinum(II) chloride

platinum(IV) chloride

platinum(II) iodide

platinum(II) hydroxide

hydrated platinum(II) hydroxide

platinum(II) monoxide, hydrated or otherwise

platinum(IV) dioxide, hydrated or otherwise

platinum(II) cyanide

platinum(II) acetylacetonate

potassiumbis(oxalato)platinate(II)

ammonium tetrachloroplatinate(II)

ammonium hexachloroplatinate(IV)

hydrated sodium tetrachloroplatinate(IV)

hexahydrated sodium hexachloroplatinate(IV)

potassium hexachloroplatinate(IV)

potassium hexacyanoplatinate(IV)

hydrated platinum(II) tetramine chloride

hydrated platinum(II) tetramine hydroxide

platinum(II) dichlorodiamine

platinum(IV) tetrachlorodiamine

platinum(II) dibromo(1,5-cyclooctadiene)

platinum(II) dichloro(1,5-cyclooctadiene)

platinum(II) dichlorobis(benzonitrile)

platinum(II) dichlorobis(pyridine)

hexahydrated chloroplatinic acid

platinum(II) bis(ethylenediamine) chloride

in the case of rhodium:

rhodium(III) bromide

anhydrous rhodium(III) chloride

hydrated rhodium(III) chloride

rhodium(III) iodide

rhodium(III) nitrate

rhodium(IV) dioxide, hydrated or otherwise

rhodium(III) sesquioxide, hydrated or otherwise

rhodium(II) acetate dimer

rhodium(III) acetylacetonate

hydrated sodium hexachlororhodate(III)

ammonium hexachlororhodate(III)

rhodium(I) acetylacetonatobis(ethylene)

potassium hexanitritorhodate(III)

in the case of osmium:

osmium(II) chloride

osmium(III) chloride

osmium(IV) chloride

osmium(IV) iodide

osmium(II) oxide

osmium(III) oxide

osmium(IV) oxide

in the case of iridium:

iridium(III) bromide

iridium(IV) bromide

iridium(II) chloride

iridium(III) chloride

iridium(IV) chloride

iridium(III) iodide

iridium(IV) iodide

hydrated iridium(II) nitrate

hydrated iridium(III) sulphate

iridium(IV) dioxide, hydrated or otherwise

irridium(III) sesquioxide, hydrated or otherwise

iridium(II) acetate

iridium(III) hexamine nitrate

iridium(III) hexamine chloride

The catalyst obtained, consisting therefore of a zeolite which is modified and used as acylation catalyst, is very easily regenerated at the end of reaction.

An “acylation reaction” is intended to mean a reaction that is well known to a person skilled in the art, which consists in reacting a preferably aromatic substrate with an acylating agent, it being possible for the reaction to be conducted in vapour or liquid phase.

After removal of the liquid phase if necessary, a hot gaseous stream of molecular oxygen or of a gas containing it is directed onto the catalyst.

This gas may be pure oxygen or oxygen diluted with an inert gas, for example nitrogen, or a rare gas, for example argon. Air is preferably used.

The gas flow rate is, for example, from 0.1 to 50 liters/hour.

The temperature of the gas flow is advantageously lower than 350° C. and is preferably between 100° C. and 250° C.

The duration of the treatment may vary widely, for example between 1 and 20 hours.

At the end of reaction a catalyst is recovered which has regained all of its initial catalytic activity.

It is therefore noted that the regeneration is performed at low temperature, in contrast to what is usually done with a simple zeolite.

An illustration of the regeneration of a modified zeolite-based catalyst is given in the acylation reaction of an aromatic ether, which follows.

Furthermore, it was found that the zeolite doped with an element of the platinum group was a very good catalyst for the reaction of acylation of an aromatic ether.

Another subject of the present invention is a process for acylation of an aromatic ether, which consists in reacting the said aromatic ether with an acylating agent, in the presence of a catalyst, and then in recovering the product formed, the said process being characterized in that the acylation reaction is conducted in the presence of an effective quantity of a zeolite modified with at least one element M₁ of the platinum group.

According to a preferred alternative form of the process of the invention the acylation of the aromatic ether is performed first of all by reacting it with an acylating agent, in the presence of a zeolite modified with a metallic element M₁ as defined and then, after reaction, the regeneration of the catalyst is carried out by heat-treating the said zeolite with a gas containing molecular oxygen.

In the following description of the present invention “aromatic ether” is intended to mean an aromatic compound in which a hydrogen atom bonded directly to the aromatic nucleus is replaced with an ether group and “aromatic compound” the conventional concept of aromaticity, as defined in the literature, especially by Jerry March, Advanced Organic Chemistry, 4th edition, John Wiley and Sons, 1992, pp. 40 et seq.

More precisely, the subject of the present invention is a process for acylation of an aromatic ether of general formula (I):

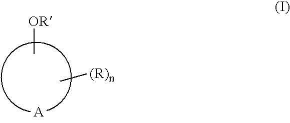

in which:

A symbolizes the residue of a ring forming all or part of a monocyclic or polycyclic aromatic carbocyclic system, the system including at least one OR′ group, it being possible for the said cyclic residue to carry one or more substituents,

R denotes one or several identical or different substituents,

R′ denotes a hydrocarbon radical containing from 1 to 24 carbon atoms, which may be a linear or branched, saturated or unsaturated, acyclic aliphatic radical, a saturated or unsaturated cycloaliphatic or monocyclic or polycyclic aromatic radical, or a linear or branched, saturated or unsaturated, aliphatic radical, carrying a cyclic substituent,

n is a number smaller than or equal to 4.

In the present text “alkoxy groups” is used, in a simplified manner, to denote the groups of the R′—O— type in which R′ has the meaning given above. R′ therefore denotes equally well a saturated or unsaturated acyclic aliphatic or cycloaliphatic or aromatic radical or a saturated or unsaturated aliphatic radical carrying a cyclic substituent.

The aromatic ether which is involved in the process of the invention corresponds to the formula (I) in which R′ denotes a linear or branched, saturated or unsaturated, acyclic aliphatic radical.

More preferably R′ denotes a linear or branched alkyl radical containing from 1 to 12 carbon atoms, preferably from 1 to 6 carbon atoms, it being optionally possible for the hydrocarbon chain to be interrupted by a heteroatom (for example oxygen) or by a functional group (for example —CO—) and/or to carry a substituent (for example a halogen or a carboxylic group).

The linear or branched, saturated or unsaturated, acyclic aliphatic radical may optionally carry a cyclic substituent. A ring is intended preferably to mean a saturated, unsaturated or aromatic carbocyclic ring, preferably cycloaliphatic or aromatic, especially cycloaliphatic containing 6 carbon atoms in the ring, or a benzene ring.

The acyclic aliphatic radical may be linked to the ring by a valency bond, a heteroatom or a functional group, and examples are given below.

The ring may be optionally substituted and, by way of examples of cyclic substituents, it is possible to envisage, among others, substituents such as R, the meaning of which is specified in the case of formula (Ia).

R′ may also denote a carbocyclic radical which is saturated or includes 1 or 2 unsaturations in the ring, generally containing from 3 to 8 carbon atoms, preferably 6 carbon atoms, in the ring, it being possible for the said ring to be substituted with substituents such as R.

R′ may also denote an aromatic, preferably monocyclic, carbocyclic radical generally containing at least 4 carbon atoms, preferably 6 carbon atoms, in the ring, it being possible for the said ring to be substituted with substituents such as R.

The process of the invention applies very particularly to the aromatic ethers of formula (I) in which R′ denotes a linear or branched alkyl radical containing from 1 to 4 carbon atoms or a phenyl radical.

Examples of preferred radicals R′ according to the invention which may be mentioned are methyl, ethyl, propyl, isopropyl, butyl, isobutyl, sec-butyl, tert-butyl and phenyl radicals.

In the general formula (I) of the aromatic ethers the residue A may denote the residue of a monocyclic aromatic carbocyclic compound containing at least 4 carbon atoms and preferably 6 carbon atoms or the residue of a polycyclic carbocyclic compound which may consist of at least 2 carbon rings which are aromatic and which form ortho- or ortho- and pericondensed systems between them or of at least 2 carbon rings of which at least one is aromatic, and which form ortho- or ortho- and pericondensed systems between them.

The residue A may carry one or more substituents on the aromatic nucleus.

Examples of substituents R are given below, but this list is not limiting in character. Any substituent whatever may be present on the ring, provided that it does not interfere with the desired product.

Since the residue A can, among other things, carry several alkoxy groups, it is possible to acylate polyalkoxylated compounds according to the process of the invention.

The process of the invention applies more particularly to the aromatic ethers of formula (Ia):

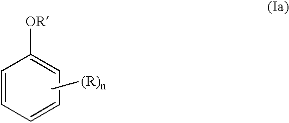

in which:

n is a number smaller than or equal to 4, preferably equal to 0, 1 or 2,

the radical R′ denotes a linear or branched alkyl radical containing from 1 to 6 carbon atoms, preferably from 1 to 4 carbon atoms, such as methyl, ethyl, propyl, isopropyl, butyl, isobutyl, sec-butyl, tert-butyl or phenyl,

the radical(s) R denote one of the following atoms or groups:

a hydrogen atom,

a linear or branched alkyl radical containing from 1 to 6 carbon atoms, preferably from 1 to 4 carbon atoms, such as methyl, ethyl, propyl, isopropyl, butyl, isobutyl, sec-butyl or tert-butyl,

a linear or branched alkoxy radical containing from 1 to 6 carbon atoms, preferably from 1 to 4 carbon atoms, such as methoxy, ethoxy, propoxy, isopropoxy, butoxy, isobutoxy, sec-butoxy or tert-butoxy radicals,

a halogen atom, preferably a fluorine, chlorine or bromine atom, or a trifluoromethyl radical,

the radicals R′ and R and the 2 successive atoms of the benzene ring can together form a ring containing from 5 to 7 atoms, optionally including another heteroatom.

When n is greater than or equal to 1 the radicals R′ and R and the 2 successive atoms of the benzene ring may be linked together by an alkylene, alkenylene or alkenylidene radical containing from 2 to 4 carbon atoms, to form a saturated, unsaturated or aromatic heterocyclic ring containing from 5 to 7 carbon atoms. One or more carbon atoms may be replaced by another heteroatom, preferably oxygen. The radicals R′ and R can thus denote a methylenedioxy or ethylenedioxy radical.

The process of the invention applies more particularly to the aromatic ethers of formula (Ia) in which n is equal to 1, the radicals R and R′ both denoting identical or different alkoxy radicals.

By way of illustration of compounds corresponding to the formula (I) there may be mentioned more particularly:

monoethers such as anisole, ethoxybenzene (phenetole), butoxybenzene, isobutoxybenzene, 2-chloroanisole, 3-chloroanisole, 2-bromoanisole, 3-bromoanisole, 2-methylanisole, 3-methylanisole, 2-ethylanisole, 3-ethylanisole, 2-isopropylanisole, 3-isopropylanisole, 2-propylanisole, 3-propylanisole, 2-allylanisole, 2-butylanisole, 3-butylanisole, 2-benzylanisole, 2-cyclohexylanisole, 1-bromo-2-ethoxybenzene, 1-bromo-3-ethoxybenzene, 1-chloro-2-ethoxybenzene, 1-chloro-3-ethoxybenzene, 1-ethoxy-2-ethylbenzene, 1-ethoxy-3-ethylbenzene, 2,3-dimethylanisole and 2,5-dimethylanisole,

diethers such as veratrole, 1,3-dimethoxybenzene, 1,2-diethoxybenzene, 1,3-diethoxybenzene, 1,2-dipropoxybenzene, 1,3-dipropoxybenzene, 1,2-methylenedioxybenzene and 1,2-ethylenedioxybenzene,

triethers such as 1,2,3-trimethoxybenzene, 1,3,5-trimethoxybenzene and 1,3,5-triethoxybenzene.

The compounds to which the process according to the invention applies in a more particularly advantageous manner are anisole and veratrole.

With regard to the acylating reactant, this corresponds more particularly to the formula (II):

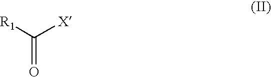

in which:

R₁ denotes:

a linear or branched, saturated or unsaturated, aliphatic radical containing from 1 to 24 carbon atoms, a saturated, unsaturated cycloaliphatic or monocyclic or polycyclic aromatic radical containing from 3 to 8 carbon atoms, or a linear or branched, saturated or unsaturated, aliphatic radical carrying a cyclic substituent,

X′ denotes:

a halogen atom, preferably a chlorine or bromine atom,

a hydroxyl group,

a radical —O—CO—R₂ with R₂, identical or different from R₁, having the same meaning as R₁, it being possible for R₁ and R₂ to form together a linear or branched, saturated or unsaturated aliphatic divalent radical containing at least 2 carbon atoms.

By cyclic substituent reference is made to what is described above.

More preferably R₁ denotes a linear or branched alkyl radical containing from 1 to 12 carbon atoms, preferably from 1 to 6 carbon atoms, it being possible for the hydrocarbon chain to be optionally interrupted by a heteroatom (for example oxygen), by a functional group (for example —CO—) and/or to carry a substituent (for example a halogen or a CF₃ group).

R₁ preferably denotes an alkyl radical containing from 1 to 4 carbon atoms, such as methyl, ethyl, propyl, isopropyl, butyl, isobutyl, sec-butyl or tert-butyl.

The radical R₁ also denotes preferably a phenyl radical which may be optionally substituted. Any substituent whatever may be present on the ring, provided that it does not interfere with the desired product.

More particular examples of substituents which may be mentioned are, especially:

a linear or branched alkyl radical containing from 1 to 6 carbon atoms, preferably from 1 to 4 carbon atoms, such as methyl, ethyl, propyl, isopropyl, butyl, isobutyl, sec-butyl or tert-butyl,

a linear or branched alkoxy radical containing from 1 to 6 carbon atoms, preferably from 1 to 4 carbon atoms, such as the methoxy, ethoxy, propoxy, isopropoxy, butoxy, isobutoxy, sec-butoxy and tert-butoxy radicals,

a hydroxyl group,

a halogen atom, preferably a fluorine, chlorine or bromine atom.

The preferred acylating agents are acid anhydrides. They correspond more particularly to the formula (II) in which R₁ and R₂ are identical and denote an alkyl radical containing from 1 to 4 carbon atoms.

When the acylating agent is an acid halide, it corresponds preferably to the formula (II) in which X′ denotes a chlorine atom and R₁ denotes a methyl or ethyl radical.

By way of illustration of acylating agents corresponding to the formula (II) there may be mentioned more particularly:

acetic anhydride,

propanoic anhydride,

isobutyric anhydride,

trifluoroacetic anhydride,

benzoic anhydride,

acetyl chloride,

monochloroacetyl chloride,

dichloroacetyl chloride,

propanoyl chloride,

isobutanoyl chloride,

pivaloyl chloride,

stearoyl chloride,

crotonyl chloride,

benzoyl chloride,

chlorobenzoyl chlorides,

p-nitrobenzoyl chloride,

methoxybenzoyl chlorides,

naphthoyl chlorides,

acetic acid.

In accordance with the invention the acylation reaction is advantageously conducted in liquid phase including the aromatic ether and the acylating agent, in the presence of the catalyst.

One of the starting reactants may be used as reaction solvent, but it is also possible to use an organic solvent.

As examples of solvents which are suitable for the present invention there may be mentioned in particular aliphatic or aromatic hydrocarbons, halogenated or otherwise, and aliphatic, cycloaliphatic or aromatic ethers.

Examples of aliphatic hydrocarbons which may be mentioned more particularly are the paraffins such as especially hexane, heptane, octane, nonane, decane, undecane, dodecane, tetradecane or cyclohexane and naphthalene and the aromatic hydrocarbons and more particularly the aromatic hydrocarbons like especially benzene, toluene, xylenes, cumene and the petroleum cuts consisting of a mixture of alkylbenzenes, especially the cuts of Solvesso® type.

Insofar as the aliphatic or aromatic halogenated hydrocarbons are concerned, it is possible to mention more particularly the perchlorinated hydrocarbons such as especially tetrachloroethylene and hexachloroethane, partially chlorinated hydrocarbons such as dichloromethane, chloroform, 1,2-dichloroethane, 1,1,1-trichloroethane, 1,1,2,2-tetrachloroethane, pentachloroethane, trichloroethylene, 1-chlorobutane and 1,2-dichlorobutane, monochlorobenzene, 1,2-dichlorobenzene, 1,3-dichlorobenzene, 1,4-dichlorobenzene, 1,2,4-trichlorobenzene or mixtures of various chlorobenzenes, bromoform, bromoethane or 1,2-dibromoethane, monobromobenzene or mixtures of monobromobenzene with one or more dibromobenzenes, and 1-bromonaphthalene.

Aliphatic, cycloaliphatic or aromatic ethers may also be employed as organic solvents and, more particularly, diethyl ether, dipropyl ether, diisopropyl ether, dibutyl ether, methyl tert-butyl ether, dipentyl ether, diisopentyl ether, ethylene glycol dimethyl ether (or 1,2-dimethoxyethane), diethylene glycol dimethyl ether (or 1,5-dimethoxy-3-oxapentane), benzyl ether, dioxane and tetrahydrofuran (THF).

Use may be made of polar aprotic solvents such as the nitro compounds like, for example, nitromethane, nitroethane, 1-nitropropane, 2-nitropropane or mixtures thereof, nitrobenzene, aliphatic or aromatic nitrites like acetonitrile, propionitrile, butanenitrile, isobutanenitrile, benzonitrile and benzyl cyanide, and tetramethylene sulphone (sulpholane).

The preferred solvents are dichloromethane, tetrachloromethane, THF and diethyl ether.

A mixture of organic solvents may also be employed.

The starting substrate is preferably employed as reaction solvent.

In a first stage of the process of the invention the acylation of the aromatic ether is conducted. According to a preferred alternative form the regeneration of the catalyst is performed in a following stage.

The ratio of the number of moles of aromatic ether to the number of moles of acylating agent may vary because the substrate may be used as reaction solvent. The ratio may thus range from 0.1 to 10 and preferably lies between 0.5 and 4.0.

The quantity of catalyst which is used in the process of the invention may vary within wide limits.

When the process is carried out noncontinuously, the catalyst may represent from 0.01 to 50%, preferably from 1.0 to 20%, by weight relative to the aromatic ether introduced.

However, if the process is carried out continuously, for example by reacting a mixture of the aromatic ether and of the acylating agent on a stationary catalyst bed, these catalyst/aromatic ether ratios are meaningless and, at a given instant there may be a weight excess of catalyst in relation to the starting aromatic ether. In this case the residence time of the flow of material over the catalyst bed varies, for example, between 5 min and 10 hours and preferably between 15 min and 3 hours, depending on the reaction temperature and the desired degree of conversion.

With regard to the quantity of organic solvent which is used, this is generally chosen so that the ratio of the number of moles of organic solvent to the number of moles of aromatic ether varies preferably between 0 and 100 and still more preferably between 0 and 50.

The temperature at which the acylation reaction is carried out depends on the reactivity of the starting substrate and that of the acylating agent.

It lies between 20° C. and 300° C., preferably between 40° C. and 200° C.

The reaction is generally conducted at atmospheric pressure, but lower or higher pressures may also be suitable. The work is done at autogenous pressure when the reaction temperature is higher than the boiling temperature of the reactants and/or of the products.

From a practical viewpoint the process may be operated noncontinuously or continuously.

According to the first alternative form there are no constraints concerning the use of the reactants. They may be introduced in any order.

After the reactants have been brought into contact the reaction mixture is heated to the desired temperature.

The other alternative form of the invention consists in conducting the reaction continuously, in a tubular reactor comprising the solid catalyst placed in a stationary bed.

The aromatic ether and the acylating agent may be introduced into the reactor separately or as a mixture.

They may also be introduced in a solvent such as mentioned above.

At the end of reaction a liquid phase is recovered including the acylated aromatic ether, which may be recovered in a conventional way, by distillation or by recrystallization from a suitable solvent, after preliminary removal of the excess reactants.

According to a preferred embodiment of the invention a regeneration of the catalyst employed is performed in a following stage of the process of the invention.

After removal of the liquid phase a hot gaseous stream of molecular oxygen or of a gas containing it is sent over the catalyst.

The regeneration of the catalyst is conducted as described above.

At the end of treatment a catalyst is recovered which has regained all its initial catalytic activity.

The process of the invention is particularly well suited to the preparation of 4-methoxyacetophenone and of 3,4-dimethoxyacetophenone, commonly called acetoveratrole, by acetylation of anisole or of veratrole respectively.

An advantage of the process of the invention is that the acylation reaction takes place without there being any O-dealkylation of the starting aromatic ether.

In addition, the presence of little ortho isomer is observed, because of the para-orienting nature of the catalyst.

The examples which follow illustrate the invention without, however, limiting it.

In the examples the yields mentioned correspond to the following definition: Yield: RY_{A.A.}=(number of moles of acylating agent which are introduced)/(number of moles of acylated aromatic compound which are formed) %

### EXAMPLE 1

Preparation of a zeolite β modified with palladium.

The following are placed in a 250-cm³ polyethylene bottle:

39.5 cm³ of a solution of palladium tetrammine hydroxide Pd(NH₃)₄(OH)₂; solution CLAL 8949 containing 15.2 g/l of palladium,

60.5 cm³ of deionized water.

20 g of a powdered zeolite β in H⁺ form, marketed by Valdor under the name (PQ)CBV 811-25 are added next.

The materials are agitated for 24 h at ambient temperature (20° C.).

The modified zeolite is separated off by centrifuging, is dried in the oven at 110° C. and is then calcined at 450° C. for 2 hours.

A zeolite containing 3% by weight of palladium is obtained.

### EXAMPLE 2

13 ml of zeolite Pd (3%)/Hβ (i.e. approximately 5 g) in the form of powder prepared according to Example 1 are introduced into a tubular reactor heated by a jacket.

The jacket is heated to 100° C. and a mixture of anisole and of acetic anhydride, used in a molar ratio of 2, is then introduced through the bottom of the reactor with the aid of an HPLC pump, at a flow rate of 0.2 ml/min.

The reaction mixture is drawn off continuously by overflowing.

The reaction yield is followed in the course of time by removing aliquots which are then analysed by vapour phase chromatography.

After 100 hours' reaction the acetonitrile yield has dropped to approximately 10%.

TABLE I
initial activity

| Time (h) | Yield (%/acetic anhydride) |
|---|---|
| 3.5 | 72 |
| 7.5 | 73 |
| 30 | 50 |
| 51.2 | 26.2 |
| 70 | 20 |
| 94.3 | 13 |
| 100 | 10 |

The reactor is drained and the catalyst is reactivated in air at 190° C. for 3 hours.

The injection of the anisole/acetic anhydride mixture (2/1) at 100° C. is then restarted.

The following performance is obtained:

TABLE II
after reactivation (in air,
3 h at 190° C.)

| Time (h) | Yield (%/acetic anhydride) |
|---|---|
| 2.6 | 73.2 |
| 11 | 58 |
| 36 | 34.5 |
| 51 | 24 |
| 70 | 20 |
| 93 | 13 |

### EXAMPLE 3

13 ml of zeolite Pd (3%)/Hβ (i.e. approximately 5 g) prepared according to Example 1, in the form of extrudates containing 40% of alumina binder, are introduced into a tubular reactor heated by a jacket.

The jacket is heated to 105° C. and a mixture of anisole and of acetic anhydride used in a molar ratio of 2 is then introduced through the bottom of the reactor with the aid of an HPLC pump, at a flow rate of 0.2 ml/min.

The reaction mixture is drawn off continuously by overflow.

The yield of the reaction is followed in the course of time by removing aliquots which are then analysed by vapour phase chromatography.

After 120 hours' reaction the yield of acetoanisole has dropped to approximately 13%.

TABLE III
initial activity

| Time (h) | Yield (%/acetic anhydride) |
|---|---|
| 4 | 70 |
| 5 | 75 |
| 30 | 40 |
| 53 | 29 |
| 70 | 24.7 |
| 100 | 23.2 |
| 120 | 13.4 |

The reactor is drained and the catalyst is reactivated in air at 180° C. for 3 hours.

The injection of the anisole/acetic anhydride (2/1) mixture at 105° C. is then restarted.

The following performance is obtained:

TABLE IV
after reactivation (in air,
3 h at 180° C.

| Time (h) | Yield (%/acetic anhydride) |
|---|---|
| 4.3 | 53 |
| 6 | 56 |
| 21.2 | 32.5 |
| 23.3 | 31.4 |

1. Process facilitating the regeneration of a catalyst based on a zeolite, employed in an acylation reaction, characterized in that the zeolite is modified by addition of an effective quantity of at least one metallic element M chosen from the elements of group 8 of the Periodic Classification of the elements.

2. Process according to claim 1, characterized in that the catalyst is a zeolite modified with the metallic element M chosen from iron, nickel, ruthenium, rhodium, palladium, osmium, iridium and platinum.

3. Process according to claim 1, characterized in that the catalyst is a zeolite modified with the metallic element M₁ chosen from ruthenium, rhodium, palladium, osmium, iridium and platinum.

4. Process according to either of claims 2 and 3, characterized in that the catalyst is a zeolite modified with the metallic element M or M₁ used in a quantity such that the percentage by weight of the metal relative to the zeolite is between 0.1% and 25%, preferably between 3% and 15%.

5. Process according to one of claims 1 to 4, characterized in that the zeolite is a natural or synthetic zeolite.

6. Process according to claim 5, characterized in that the zeolite is a natural zeolite chosen from chabazite, clinoptilolite, erionite, mordenite, phillipsite and offretite.

7. Process according to claim 5, characterized in that the zeolite is a synthetic zeolite chosen from:
synthetic zeolites with a one-dimensional network such as zeolite ZSM-4, zeolite L, zeolite ZSM-12, zeolite ZSM-22, zeolite ZSM-23 and zeolite ZSM-48,
zeolites with a two-dimensional network, such as zeolite β, mordenite and ferrierite,
zeolites with a three-dimensional network such as zeolite Y, zeolite X, zeolite ZSM-5, zeolite ZSM-11 and offretite.

8. Process according to claim 7, characterized in that the zeolite is a zeolite β and Y.

9. Process according to claims 1 to 8, characterized in that the metallic element(s) M or M₁ are deposited on the zeolite especially by precipitation or by impregnation by a dry or wet route.

10. Process according to one of claims 1 to 9, characterized in that the regeneration of the catalyst is conducted by sending a hot gaseous stream of molecular oxygen or of a gas containing it over the catalyst.

11. Process according to claim 10, characterized in that pure oxygen or oxygen diluted with an inert gas, preferably air, is used.

12. Process according to either of claims 10 and 11, characterized in that the temperature of the gaseous stream is lower than 350° C. and is preferably between 100° C. and 250° C.

13. Process according to one of claims 1 to 12, characterized in that a catalyst which can be reemployed in an acylation reaction is recovered.

14. Process for acylation of an aromatic ether, which consists in reacting an aromatic ether with an acylating agent, in the presence of a catalyst, and in then recovering the product formed, the said process being characterized in that the acylation reaction is conducted in the presence of an effective quantity of a zeolite modified with at least one element M₁ of the platinum group.

15. Process according to claim 14, characterized in that the aromatic ether corresponds to the general formula (I): 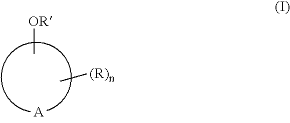
in which:
A symbolizes the residue of a ring forming all or part of a monocyclic or polycyclic aromatic carbocyclic system, the system including at least one OR′ group, it being possible for the said cyclic residue to carry one or more substituents,
R denotes one or a number of identical or different substituents,
R′ denotes a hydrocarbon radical containing from 1 to 24 carbon atoms, which may be a linear or branched, saturated or unsaturated, acyclic aliphatic radical, a saturated or unsaturated cycloaliphatic or monocyclic or polycyclic aromatic radical, or a linear or branched, saturated or unsaturated, aliphatic radical carrying a cyclic substituent,
n is a number smaller than or equal to 4.

16. Process according to either of claims 14 and 15, characterized in that the aromatic ether corresponds to the general formula (I) in which R′ denotes:
a linear or branched, saturated or unsaturated, acyclic aliphatic radical, preferably a linear or branched alkyl radical containing from 1 to 12 carbon atoms, preferably from 1 to 6 carbon atoms, it being possible for the hydrocarbon chain to be optionally interrupted by a heteroatom, a functional group and/or to carry a substituent,
a linear or branched, saturated or unsaturated, acyclic aliphatic radical carrying an optionally substituted cyclic substituent, it being possible for the said acyclic radical to be linked to the ring by a valency bond, a heteroatom or a functional group,
a carbocyclic radical which is saturated or includes 1 or 2 unsaturations in the ring, generally containing from 3 to 8 carbon atoms, preferably 6 carbon atoms in the ring, it being possible for the said ring to be substituted,
a preferably monocyclic aromatic carbocyclic radical generally containing at least 4 carbon atoms, preferably 6 carbon atoms, in the ring, it being possible for the said ring to be substituted.

17. Process according to claim 14, characterized in that the aromatic ether corresponds to the general formula (I) in which R′ denotes a linear or branched alkyl radical containing from 1 to 4 carbon atoms, preferably a methyl radical or a phenyl radical.

18. Process according to one of claims 14 to 17, characterized in that the aromatic ether corresponds to the general formula (I) in which the residue A denotes the residue of a monocyclic aromatic carbocyclic compound containing at least 4 carbon atoms and preferably 6 carbon atoms or the residue of a polycyclic carbocyclic compound, it being possible for the residue A to carry one or more substituents on the aromatic nucleus.

19. Process according to one of claims 14 to 18, characterized in that the aromatic ether corresponds to the formula (Ia): 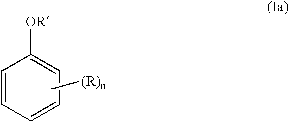
in which:
n is a number smaller than or equal to 4, preferably equal to 0, 1 or 2,
the radical R′ denotes a linear or branched alkyl radical containing from 1 to 6 carbon atoms, preferably from 1 to 4 carbon atoms, such as methyl, ethyl, propyl, isopropyl, butyl, isobutyl, sec-butyl, tert-butyl or a phenyl radical,
the radical(s) R denote one of the following atoms or groups:
a hydrogen atom,
a linear or branched alkyl radical containing from 1 to 6 carbon atoms, preferably from 1 to 4 carbon atoms, such as methyl, ethyl, propyl, isopropyl, butyl, isobutyl, sec-butyl or tert-butyl,
a linear or branched alkoxy radical containing from 1 to 6 carbon atoms, preferably from 1 to 4 carbon atoms, such as methoxy, ethoxy, propoxy, isopropoxy, butoxy, isobutoxy, sec-butoxy or tert-butoxy radicals,
a halogen atom, preferably a fluorine, chlorine or bromine atom, or a trifluoromethyl radical,
the radicals R′ and R and the 2 successive atoms of the benzene ring can together form a ring containing from 5 to 7 atoms, optionally including another heteroatom.

20. Process according to claim 19, characterized in that the aromatic ether corresponds to the formula (Ia) in which n is greater than or equal to 1, the radicals R′ and R and the 2 successive atoms of the benzene ring may be linked together by an alkylene, alkenylene or alkenylidene radical containing from 2 to 4 carbon atoms to form a saturated, unsaturated or aromatic heterocyclic ring containing from 5 to 7 carbon atoms, in which one or more carbon atoms may be replaced by a heteroatom, preferably oxygen, the radicals R′ and R preferably forming a methylenedioxy or ethylenedioxy radical.

21. Process according to either of claims 19 and 20, characterized in that the aromatic ether corresponds to the formula (Ia) in which n is equal to 1, the radicals R and R′ both denoting identical or different alkoxy radicals.

22. Process according to claim 14 and 15, characterized in that the aromatic ether is anisole or veratrole.

23. Process according to one of claims 14 to 22, characterized in that the acylating agent corresponds to the formula (II): 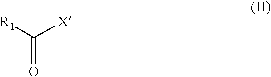
in which:
R₁ denotes:
a linear or branched, saturated or unsaturated, aliphatic radical containing from 1 to 24 carbon atoms, a saturated, unsaturated cycloaliphatic or a monocyclic or polycyclic aromatic radical containing from 3 to 8 carbon atoms, or a linear or branched, saturated or unsaturated, aliphatic radical carrying a cyclic substituent,
X′ denotes:
a halogen atom, preferably a chlorine or bromine atom,
a hydroxyl group,
a radical —O—CO—R₂ with R₂, identical or different from R₁ having the same meaning as R₁, it being possible for R₁ and R₂ to form together a linear or branched, saturated or unsaturated aliphatic divalent radical containing at least 2 carbon atoms.

24. Process according to claim 23, characterized in that the acylating agent corresponds to the formula (II) in which X′ denotes a chlorine atom and R₁ denotes a linear or branched alkyl radical containing from 1 to 12 carbon atoms, it being possible for the hydrocarbon chain to be optionally interrupted by a heteroatom or by a functional group or to carry a substituent, R₁ denotes an optionally substituted phenyl radical, or X′ denotes a radical —O—CO—R₂ in which R₁ and R₂ are identical and denote an alkyl radical containing from 1 to 4 carbon atoms.

25. Process according to claim 23 and 24, characterized in that the acylating agent is chosen from:
acetic anhydride,
propanoic anhydride,
isobutyric anhydride,
trifluoroacetic anhydride,
benzoic anhydride,
acetyl chloride,
monochloroacetyl chloride,
dichloroacetyl chloride,
propanoyl chloride,
isobutanoyl chloride,
pivaloyl chloride,
stearoyl chloride,
crotonyl chloride,
benzoyl chloride,
chlorobenzoyl chlorides,
p-nitrobenzoyl chloride,
methoxybenzoyl chlorides,
naphthoyl chlorides,
acetic acid.

26. Process according to one of claims 14 to 25, characterized in that the catalyst is a zeolite modified with the metallic element M₁ chosen from ruthenium, rhodium, palladium, osmium, iridium and platinum.

27. Process according to one of claims 14 to 26, characterized in that the catalyst is a zeolite modified with the metallic element M₁ used in a quantity such that the percentage by weight of the metal M₁ relative to the zeolite is between 0.1% and 25%, preferably between 3% and 15%.

28. Process according to one of claims 14 to 27, characterized in that the zeolite is a natural or synthetic zeolite.

29. Process according to claim 28, characterized in that the zeolite is a natural zeolite chosen from chabazite, clinoptilolite, erionite, mordenite, phillipsite and offretite.

30. Process according to claim 28, characterized in that the zeolite is a synthetic zeolite chosen from:
synthetic zeolites with a one-dimensional network such as zeolite ZSM-4, zeolite L, zeolite ZSM-12, zeolite ZSM-22, zeolite ZSM-23 and zeolite ZSM-48,
zeolites with a two-dimensional network, such as zeolite β, mordenite and ferrierite,
zeolites with a three-dimensional network such as zeolite Y, zeolite X, zeolite ZSM-5, zeolite ZSM-11 and offretite.

31. Process according to claim 29, characterized in that the zeolite is a zeolite β and Y.

32. Process according to one of claims 14 to 31, characterized in that an organic solvent is used, chosen from optionally halogenated, preferably chlorinated aliphatic and/or aromatic hydrocarbons, aliphatic, cycloaliphatic or aromatic ethers, nitro compounds, aliphatic or aromatic nitrites and tetramethylene sulphone.

33. Process according to one of claims 14 to 32, characterized in that the ratio of the number of moles of aromatic ether to the number of moles of acylating agent varies between 0.1 and 10 and preferably lies between 0.5 and 4.

34. Process according to one of claims 14 to 33, characterized in that in a noncontinuous method of operation the quantity of catalyst represents from 0.01 to 50%, preferably from 1.0 to 20% by weight relative to the aromatic ether introduced and, in a continuous method of operation, the residence time of the flow of material on the catalyst bed varies between 5 min and 10 hours and preferably between 15 min and 3 hours.

35. Process according to one of claims 14 to 34, characterized in that the temperature at which the acylation reaction is carried out lies between 20° C. and 300° C., preferably between 40° C. and 200° C.

36. Process according to one of claims 14 to 35, characterized in that the regeneration of the catalyst is performed after removal of the liquid phase, according to the process described in one of claims 10 to 12.

37. Process according to one of claims 14 to 36, characterized in that a catalyst which can be reemployed in the acylation process of the invention as described in one of claims 14 to 35 is recovered.

38. Catalyst for acylation of an aromatic ether, including a zeolite modified with an element of the platinum group.

39. Catalyst for acylation of anisole, including a zeolite β modified with an element of the platinum group.
